(19) United States
(12) Patent Application Publication
(10) Pub. No.: US 2013/0211176 A1
(43) Pub. Date: Aug. 15, 2013

(54) ENHANCED ABLATION APPARATUS

(71) Applicant: Nagy Habib, London (GB)

(72) Inventor: Nagy Habib, London (GB)

(73) Assignee: EMcision Limited, London (GB)

(21) Appl. No.: 13/806,073

(22) PCT Filed: Jun. 24, 2011
(86) PCT No.: PCT/GB11/51204

§ 371 Date: Apr. 24, 2013

(30) Foreign Application Priority Data
Jun. 24, 2010 (GB) 1010641.7
Nov. 4, 2010 (GB) 1018621.1
Mar. 4, 2011 (GB) 1103779.3

(51) Int. Cl. A61B 18/14; A61N 5/10; A61N 7/00

(52) U.S. Cl. CPC A61B 18/1492; A61N 7/00; A61N 5/1001
USPC 600/3; 606/41; 606/33; 606/169

(57) ABSTRACT

Apparatus and methods suitable for causing tissue ablation at a specified therapeutic site in the body of a patient. The apparatus comprises an ablation device having a distal end and a proximal end and a central lumen extending along its length, the distal end comprising at least one energy delivery element suitable for causing tissue ablation. A penetrating member having a distal end and a proximal end, the distal end comprising a sharp tip suitable for piercing tissue and creating a channel for the device in the tissue, is coaxially positioned within the central lumen of the ablation device and is capable of being advanced distally out of the central lumen of the device and retracted back to within the central lumen of the device. The apparatus may further comprise an endoscope for delivery of the device to the site of treatment.

### FIELD

The invention relates to apparatus and methods for performing endoscopic and percutaneous interventional surgery. In particular the invention relates to apparatus and methods for ablating lesions in the body.

### BACKGROUND

Lesions are any type of abnormal tissue in or on the body of an organism, which has usually been damaged by disease or trauma. Lesions, such those resulting from tumours, are a major cause of death and morbidity. Solid tumours within the body may be as a result of primary cancers or secondary tumours following metastasis of the primary cancer. Cancerous tumours are an example of abnormal tissue and typically the tissue surrounding the tumour will also be damaged.

It is often desirous to remove the abnormal tissue from the body. Conventional surgical intervention, such as via laparotomy, can be highly traumatic to the patient and increases the risk of major blood loss and infection. In vulnerable patients who are already weakened by chemotherapy, additional palliative treatments may not be an option where conventional large scale surgery is the only known route available. Hence, there has been an increased need for less invasive laparoscopic and endoscopic procedures where possible.

Percutaneous surgical procedures involve insertion of a therapeutic probe, typically a catheter mounted on a guidewire, through an incision made in the skin of the patient. The probe can be guided to a therapeutic site in the body via the circulatory system of arteries and veins (i.e. endovascular surgery), thereby reducing the need to cause more extensive trauma to the patient by adopting more traditional open surgical techniques.

Endoscopic surgical procedures involve insertion of an endoscope directly into an organ of the body to examine the interior of a hollow organ, vessel or cavity of the body. Endoscopy can involve, for example, the gastrointestinal tract (including the esophagus, the stomach and duodenum, the small intestine, the large intestine\colon, the bile duct, the rectum and the anus), the respiratory tract, the urinary tract, and the female reproductive system (including the cervix, the uterus and the fallopian tubes). Scarless operations can be performed using a surgical technique known as natural orifice transluminal endoscopic surgery (NOTES) in which an endoscope is passed through a natural orifice, such as the mouth, then through an incision in the stomach, bladder or colon, for example, thus avoiding any external incisions or scars.

Treatment probes, such as ablation catheters, can be inserted through the lumen of an endoscope to treat lesions in the body.

An example of a treatment probe for RF ablation of tissue and which is delivered to the site of therapy via an endoscope is described in EP 1,870,051. The device comprises a needle pipe, housed within a guide tube, which is used to puncture tissue at the site of therapy. A stylet, housed in the lumen of the needle pipe and protruding slightly from the distal end of the needle pipe, prevents the guide tube from being damaged by the sharp distal end of the needle pipe. Once the needle pipe has punctured tissue at the site of therapy, it is removed from the guide tube and a treatment probe comprising electrodes at its distal end is inserted through the lumen of the guide tube and into the site of treatment through the puncture hole.

The disadvantage of this type of device is that the needle pipe and stylet need to be completely removed from the guide tube after puncture of the tissue has occurred in order to allow the subsequent advancement of the treatment probe through the guide tube to the site of therapy. In the time that this takes the site of the puncture may have moved or additional complications may have occurred.

There exists a need for apparatus and methods which can be used to ablate lesions in the body via an endoscopic or percutaneous route in a simple, direct and effective manner.

### SUMMARY

In a first aspect the invention provides an apparatus suitable for causing tissue ablation at a specified therapeutic site in the body of a patient, comprising:
an ablation device having a distal end and a proximal end and a central lumen extending along its length, the distal end comprising at least one energy delivery element suitable for causing tissue ablation; and
a penetrating member having a distal end and a proximal end, the distal end comprising a sharp tip suitable for piercing tissue and creating a channel for the device in the tissue,
wherein the penetrating member is coaxially positioned within the central lumen of the device and is capable of being advanced distally out of the central lumen of the device and retracted back to within the central lumen of the device.

Suitably, the at least one energy delivery element may be located at the distal tip of the device.

The at least one energy delivery element may be selected from: a monopolar radiofrequency electrode arrangement; a bipolar radiofrequency electrode arrangement; a plurality of radiofrequency electrodes; a microwave energy source; an ultrasound energy source; an irreversible electroporation energy source; and an electrical current energy source. Preferably, the at least one energy delivery element comprises a bipolar radiofrequency electrode arrangement, comprising a first electrode located at the distal end of the device and a second electrode located at a position proximally to the first electrode.

Optionally, the penetrating member may also comprise at least one energy delivery element at its distal end. Suitably, the at least one energy delivery element may be located at the distal tip of the penetrating member. The at least one energy delivery element may be selected from: a monopolar radiofrequency electrode arrangement; a bipolar radiofrequency electrode arrangement; a plurality of radiofrequency electrodes; a microwave energy source; an ultrasound energy source; an irreversible electroporation energy source; and an electrical current energy source.

Suitably, the apparatus may be slidably positioned within a central lumen of an endoscope for endoscopic delivery of the apparatus to the site of treatment. Optionally, the endoscope comprises an ultrasonic transducer at its distal end.

The apparatus may further comprise at least one enhanced ultrasound reflection surface, suitably located on the surface of the device, and/or the surface of the at least one energy delivery element and/or the surface of the penetrating member.

Optionally, the penetrating member comprises a lumen and/or a groove that runs longitudinally along its length. A guidewire may be located within the lumen or groove of the penetrating member to assist with positioning of the penetrating member.

Suitably, the device and/or penetrating member may comprise means for emitting local radiotherapy at its distal end. Preferably, this comprises an iridium-192 impregnated member that is housed within the central lumen of the device or within a lumen or groove comprised within the penetrating member, and wherein the iridium-192 impregnated member can be advanced beyond the distal tip of the apparatus so as to expose the specified therapeutic site to local radiotherapy.

In a second aspect the invention provides an apparatus suitable for the endoscopic ablation of tissue at a specified therapeutic site in the body of a patient, comprising:
an endoscope having a distal end and a proximal end, wherein the endoscope comprises a first central lumen;
an ablation device having a distal end and a proximal end and a second central lumen extending along its length, the distal end comprising at least one energy delivery element suitable for inducing tissue ablation; and
a penetrating member having a distal end and a proximal end, the distal end comprising a sharp tip suitable for piercing tissue and creating a channel for the device in the tissue,
wherein the device is capable of being coaxially positioned within the first central lumen of the endoscope and the penetrating member is capable of being coaxially positioned within the second central lumen of the device, and wherein the device is capable of being advanced distally out of the first central lumen of the endoscope and retracted back to within the first central lumen of the endoscope and the penetrating member is capable of being advanced distally out of the second central lumen of the device and retracted back to within the second central lumen of the device.

Optionally, the endoscope comprises an ultrasound transducer at its distal end.

Suitably, the at least one energy delivery element may be located at the distal tip of the device. The at least one energy delivery element may be selected from: a monopolar radiofrequency electrode arrangement; a bipolar radiofrequency electrode arrangement; a plurality of radiofrequency electrodes; a microwave energy source; an ultrasound energy source; an irreversible electroporation energy source; and an electrical current energy source. Preferably, the at least one energy delivery element comprises a bipolar radiofrequency electrode arrangement, comprising a first electrode located at the distal end of the device and a second electrode located at a position proximally to the first electrode.

The penetrating member may also comprise at least one energy delivery element at its distal end. Suitably, the at least one energy delivery element may be located at the distal tip of the penetrating member. The at least one energy delivery element may be selected from: a monopolar radiofrequency electrode arrangement; a bipolar radiofrequency electrode arrangement; a plurality of radiofrequency electrodes; a microwave energy source; an ultrasound energy source; an irreversible electroporation energy source; and an electrical current energy source.

Optionally, the apparatus may comprise at least one enhanced ultrasound reflection surface. Suitably, the at least one enhanced ultrasound reflection surface is located on the surface of the device, and/or the surface of the at least one energy delivery element, and/or the surface of the penetrating member.

Suitably, the penetrating member may comprise a lumen or a groove that runs longitudinally along its length. A guidewire may be located within the lumen or groove of the penetrating member to assist with positioning of the penetrating member.

Optionally, the device and/or penetrating member may further comprise means for emitting local radiotherapy at its distal end, preferably comprising an iridium-192 impregnated member that is housed within the second central lumen of the device or within a lumen or groove comprised within the penetrating member, and wherein the iridium-192 impregnated member can be advanced beyond the distal tip of the apparatus so as to expose the specified therapeutic site to local radiotherapy.

In a third aspect the invention provides an apparatus suitable for causing tissue ablation at a specified therapeutic site in the body of a patient, comprising:
a catheter having a distal end and a proximal end and a central lumen extending along its length; and
an ablation device having a distal end and a proximal end, the distal end comprising at least one energy delivery element suitable for inducing tissue ablation;
   wherein the ablation device is capable of being coaxially positioned within the central lumen of the catheter and is capable of being advanced distally out of the central lumen of the catheter and retracted back to within the central lumen of the catheter.

Suitably, the catheter comprises a penetrating member having a distal end comprising a sharp tip suitable for penetrating tissue.

The device may be slidably positioned within the central lumen of the catheter.

Suitably, the ablation device comprises a catheter, such as an ultra-thin catheter, or a guidewire.

Optionally, the ablation device comprises an elongate body including a conductive core about which is located an insulating layer along at least a portion of the elongate body.

Suitably, the at least one energy delivery element may be located at the distal tip of the device. The at least one energy delivery element may be selected from: a monopolar radiofrequency electrode arrangement; a bipolar radiofrequency electrode arrangement; a plurality of radiofrequency electrodes; a microwave energy source; an ultrasound energy source; an irreversible electroporation energy source; and an electrical current energy source. Preferably, the at least one energy delivery element comprises a bipolar radiofrequency electrode arrangement comprising a first electrode located at the distal end of the device and a second electrode located at a position proximally to the first electrode.

Optionally, the catheter may comprise at least one energy delivery element at its distal end. Suitably, the at least one energy delivery element may be located at the distal tip of the catheter. The at least one energy delivery element may selected from: a monopolar radiofrequency electrode arrangement; a bipolar radiofrequency electrode arrangement; a plurality of radiofrequency electrodes; a microwave energy source; an ultrasound energy source; an irreversible electroporation energy source; and an electrical current energy source.

Suitably, the apparatus may be slidably positioned within a central lumen of an endoscope for endoscopic delivery of the apparatus to the site of treatment. Optionally, the endoscope comprises an ultrasonic transducer at its distal end.

The apparatus may further comprise at least one enhanced ultrasound reflection surface. Suitably, the at least one enhanced ultrasound reflection surface is located on the surface of the device, and/or the surface of the at least one energy delivery element, and/or the surface of the catheter.

Optionally, the device and/or catheter may comprise means for emitting local radiotherapy at its distal end. Suitably, the means for emitting local radiotherapy comprises an iridium-192 impregnated member that is housed within the central lumen of the device or within a lumen or groove comprised within the catheter, and wherein the iridium-192 impregnated member can be advanced beyond the distal tip of the apparatus so as to expose the specified therapeutic site to local radiotherapy.

In a fourth aspect the invention provides an apparatus suitable for causing tissue ablation at a specified therapeutic site in the body of a patient, comprising:
an endoscope having a distal end and a proximal end, wherein the endoscope comprises a first central lumen;
a catheter having a distal end and a proximal end and a second central lumen extending along its length; and
an ablation device having a distal end and a proximal end, the distal end comprising at least one energy delivery element suitable for inducing tissue ablation;
   wherein the catheter is capable of being coaxially positioned within the first central lumen of the endoscope and the ablation device is capable of being coaxially positioned within the second central lumen of the catheter, and wherein the catheter is capable of being advanced distally out of the first central lumen of the endoscope and retracted back to within the first central lumen of the endoscope, and the ablation device is capable of being advanced distally out of the second central lumen of the catheter and retracted back to within the second central lumen of the catheter.

Suitably, the catheter comprises a penetrating member having a distal end comprising a sharp tip suitable for penetrating tissue.

The device may be slidably positioned within the second central lumen of the catheter and the catheter may slidably positioned within the first central lumen of the endoscope.

Suitably, the ablation device comprises a catheter, such as an ultra-thin catheter, or a guidewire.

Optionally, the ablation device comprises an elongate body including a conductive core about which is located an insulating layer along at least a portion of the elongate body.

Suitably, the at least one energy delivery element may be located at the distal tip of the device. The at least one energy delivery element may be selected from: a monopolar radiofrequency electrode arrangement; a bipolar radiofrequency electrode arrangement; a plurality of radiofrequency electrodes; a microwave energy source; an ultrasound energy source; an irreversible electroporation energy source; and an electrical current energy source. Preferably, the at least one energy delivery element comprises a bipolar radiofrequency electrode arrangement comprising a first electrode located at the distal end of the device and a second electrode located at a position proximally to the first electrode.

The catheter may also comprise at least one energy delivery element at its distal end. Suitably, the at least one energy delivery element may be located at the distal tip of the catheter. The at least one energy delivery element may be selected from the: a monopolar radiofrequency electrode arrangement; a bipolar radiofrequency electrode arrangement; a plurality of radiofrequency electrodes; a microwave energy source; an ultrasound energy source; an irreversible electroporation energy source; and an electrical current energy source.

Optionally, the endoscope may comprise an ultrasonic transducer at its distal end.

The apparatus may comprise at least one enhanced ultrasound reflection surface. Suitably, the at least one enhanced ultrasound reflection surface may be located on the surface of the device, and/or the surface of the at least one energy delivery element, and/or the surface of the catheter.

Optionally, the device and/or catheter may compris means for emitting local radiotherapy at its distal end. Suitably, the means for emitting local radiotherapy comprises an iridium-192 impregnated member that is housed within the central lumen of the device or within a lumen or groove comprised within the catheter, and wherein the iridium-192 impregnated member can be advanced beyond the distal tip of the apparatus so as to expose the specified therapeutic site to local radiotherapy.

In a fifth aspect the invention provides an apparatus suitable for causing tissue ablation at a specified therapeutic site in the body of a patient, comprising:
an endoscope having a distal end and a proximal end, wherein the endoscope comprises a central lumen; and
an ablation device having a distal end and a proximal end, the distal end comprising at least one energy delivery element suitable for inducing tissue ablation;
   wherein the ablation device is capable of being coaxially positioned within the central lumen of the endoscope and is capable of being advanced distally out of the central lumen of the endoscope and retracted back to within the central lumen of the endoscope.

In a sixth aspect the invention provides a method of ablating tissue at a specified therapeutic site in the body of a patient using the apparatus of the second embodiment as described above, comprising:
advancing the endoscope along a hollow organ towards the site of treatment until the distal end of the endoscope is positioned close to the site of treatment;
advancing the distal end of the penetrating member out of the distal end of the ablation device and beyond the distal end of the endoscope so as to penetrate the tissue wall of the hollow organ;
advancing the distal end of the ablation device out of the distal end of the endoscope and through the tissue wall of the hollow organ until the at least one heating element is located close to or at the site of treatment;
activating the at least one energy delivery element to cause tissue ablation at the site of treatment; and
withdrawing the apparatus from the body after tissue ablation is complete.

In a seventh aspect the invention provides a method of ablating tissue at a specified therapeutic site in the body of a patient using the apparatus of the fourth embodiment as described above, comprising:
advancing the endoscope along a hollow organ towards the site of treatment until the distal end of the endoscope is positioned close to the site of treatment;
advancing the distal end of the catheter out of the distal end of the endoscope so as to penetrate the tissue wall of the hollow organ;
advancing the distal end of the ablation device out of the distal end of the catheter and through the tissue wall of the hollow organ until the at least one heating element is located close to or at the site of treatment;
activating the at least one energy delivery element to cause tissue ablation at the site of treatment; and
withdrawing the apparatus from the body after tissue ablation is complete.

### DRAWINGS

In order that the invention may be more readily understood, reference will now be made, by way of example, to the accompanying drawings in which:

FIG. 1 shows a diagrammatic side view of an embodiment of the invention in which the ablation device comprises a catheter having a bipolar RF electrode arrangement at its distal end and a user control hub at its proximal end. A penetrating member is housed in the lumen of the ablation device.

FIG. 2 shows a cross-sectional diagrammatic side view of the distal end of the ablation device of the invention as shown in FIG. 1.

FIG. 3 shows a diagrammatic side view of the field of imaging at the distal end of an ultrasonic endoscope for use with an ablation device of the invention.

FIG. 4 shows a cross-sectional diagrammatic side view of the ultrasonic endoscope of FIG. 3 in which the ablation device of the invention as shown in FIG. 1 is positioned in the lumen of the endoscope as it would be during the insertion phase. The penetrating member is shown in the retracted position housed within the lumen of the ablation device.

FIG. 5 shows a cross-sectional diagrammatic side view of the ultrasonic endoscope of FIG. 3 in which the ablation device of the invention as shown in FIG. 1 is positioned in the lumen of the endoscope. The penetrating member is shown in an advanced position, whereby the distal end of the penetrating member extends beyond the distal end of the ablation device, to enable puncture of adjacent tissue.

FIG. 6 shows a diagrammatic side view of the ultrasonic endoscope of FIG. 3 in which the ablation device of the invention as shown in FIG. 1 is positioned in the lumen of the endoscope as it would be during the therapy phase. The ablation device is shown in an advanced position.

FIG. 7 shows a cross-sectional diagrammatic side view of the user control hub at the proximal end of the ablation device of the invention as shown in FIG. 1.

FIG. 8 shows a diagrammatic side view of an embodiment of the invention in which the ablation device comprises a catheter having a bipolar RF electrode arrangement at its distal end and a user control hub at its proximal end. A penetrating member is housed in the lumen of the ablation device and a guidewire is housed within the lumen of the penetrating member. The penetrating member is shown in the retracted position.

FIGS. 9 (a) and (b) show a diagrammatic side view of the invention of FIG. 8 in which the penetrating member is shown in an advanced position.

FIG. 10 shows a cross-sectional diagrammatic side view of the user control hub of the invention of FIG. 8.

FIG. 11 shows a diagrammatic side view of an embodiment of the invention in which the ablation device comprises a curved catheter. The ablation device is positioned in the lumen of the endoscope as it would be during the insertion phase.

FIG. 12 shows a diagrammatic side view of the ablation device of the invention as shown in FIG. 11 in which the penetrating member is shown in an advanced position, whereby the distal end of the penetrating member extends beyond the distal end of the ablation device, to enable puncture of adjacent tissue.

FIG. 13 shows a diagrammatic side view of the ablation device of the invention as shown in FIG. 11 in which the ablation device is shown in an advanced position as it would be during the therapy phase and the bipolar electrode arrangement is positioned at the desired site of treatment. A guidewire is housed in the lumen of the penetrating member.

FIG. 14(a) shows a diagrammatic side view of the heating zone (shown by broken lines) generated by a bipolar electrode arrangement on the ablation device of the invention. FIG. 14(b) shows a diagrammatic side view of the heating zone generated when an additional electrode is located on the penetrating member. Optional ultrasound reflective surfaces are shown on the body of the ablation device.

FIG. 15 shows a diagrammatic side view of an embodiment of the invention in which the ablation device comprises areas of increased ultrasound reflection on the surface of the ablation device, adjacent to and between the bipolar electrodes, and the penetrating member.

FIG. 16 shows a cross-sectional diagrammatic side view of a surface coating layer of the invention as shown in FIG. 15 in which the area of increased ultrasound reflection comprises a coating of gas-filled micro-balloons in a matrix.

FIG. 17 shows a cross-sectional diagrammatic side view of a surface coating layer of the invention as shown in FIG. 15 in which the area of increased ultrasound reflection comprises uneven surface globules.

FIG. 18 shows a cross-sectional diagrammatic side view of a surface coating layer of the invention as shown in FIG. 15 in which the area of increased ultrasound reflection comprises gas pockets trapped in a coating.

FIG. 19 shows a cross-sectional diagrammatic side view of an embodiment of the invention in which the ablation device comprises areas of increased ultrasound reflection on the bipolar electrode arrangement. A hollow micro-fibre is interspersed between the conductor of the electrode to improve ultrasound echo.

FIG. 20 shows a diagrammatic side view of the invention as shown in FIG. 15 in which the area of increased ultrasound reflection comprises a piezoelectric material.

FIG. 21 shows a diagrammatic side view of an embodiment of the invention in which the ablation device is positioned in an ultrasonic endoscope having a motion detector located at its proximal end. ‘d’ is the distance that the ablation device protrudes from the distal end of the endoscope.

FIGS. 22 (a) and (b) show further ablation devices of the invention.

FIG. 23 shows an arrangement of the ablation devices of the invention of FIGS. 22 (a) and (b).

FIG. 24 shows placement of the ablation devices of FIGS. 22 (a) and (b) coaxially within the lumen of a penetrating member.

### DETAILED DESCRIPTION

Unless stated otherwise the terms used herein have the same meanings as those understood by a person of appropriate skill in the art.

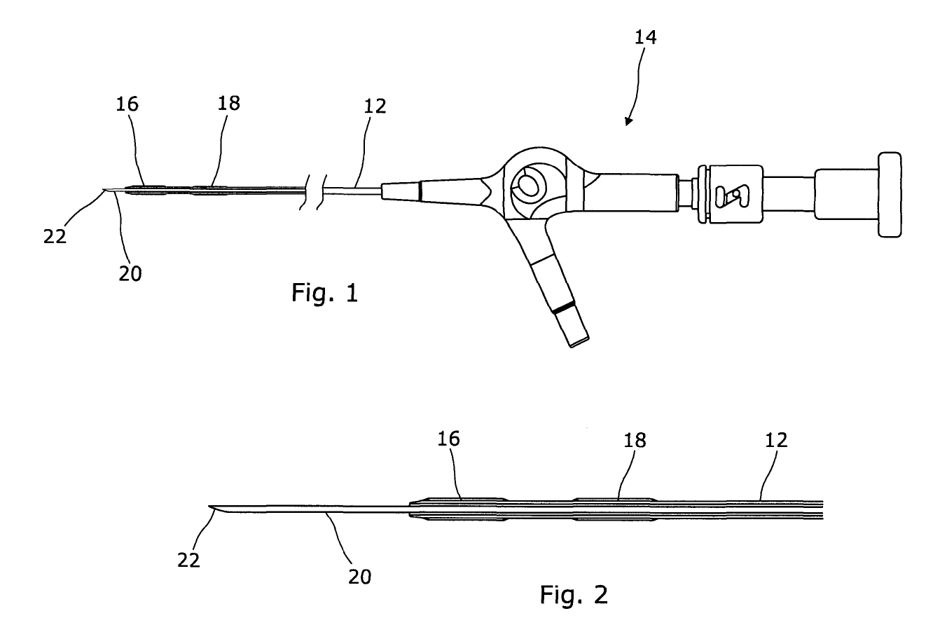

An embodiment of the invention is shown in FIGS. 1 and 2. The ablation device (also referred to as the device) comprises an elongate catheter 12 including a proximal end 14, where control of the device is administered by the user, and a distal end having a bipolar radio-frequency (RF) electrode arrangement including a distal electrode 16 and a proximal electrode 18. The distal end of the catheter is typically located at the site within the body of the patient adjacent to or proximate to where therapy is to be administered. The distal end of the device includes the distal tip (which is synonymous with the distal terminus of the device) and the area close to or adjacent to the distal tip.

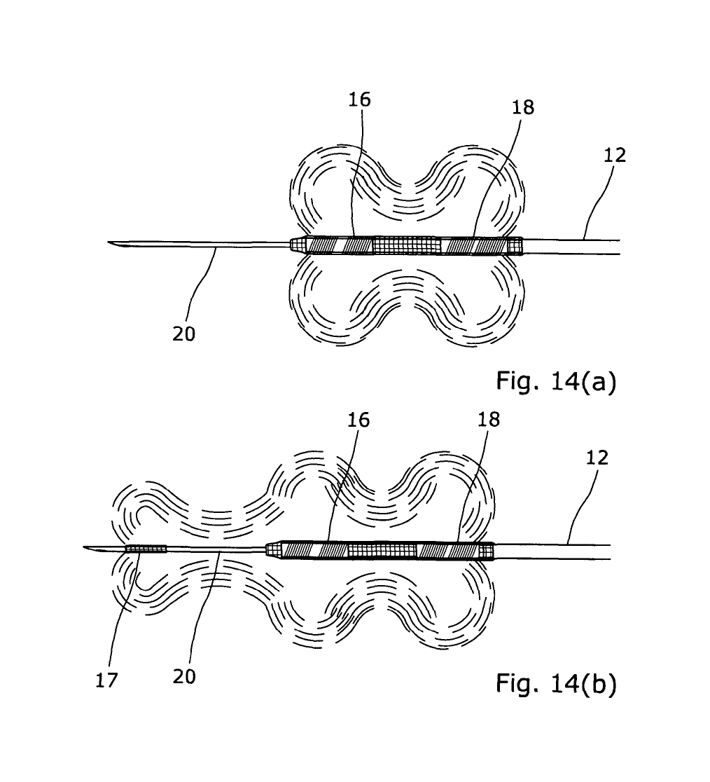

The electrodes 16 and 18 are connected to opposite polarities of an RF energy source. In use, RF current flows between the electrodes 16 and 18 and, depending upon the distance between the electrodes, results in a controlled heating zone between the electrodes which is used to ablate surrounding tissue at the site of treatment (see FIG. 14(a)). In the event that substantive tissue ablation is not required the device can simply deliver energy (e.g. RF energy, resistive heating energy, microwave energy, ultrasound energy or irreversible electroporation energy as described in further detail below) to the site of treatment, e.g. in an amount that is not high enough to cause total destruction/ablation of the surrounding tissue.

The catheter 12 has a central lumen 13 which houses a piercing or penetrating member 20, suitably a stylet, a trocar or a needle. The penetrating member 20 is a probe having a sharpened or pointed distal tip or terminus for piercing tissue and creating a channel and can be provided with or without a lumen, i.e. it can be hollow or solid. Alternatively, the penetrating member can be occupy the central lumen 13 entirely or only partially. For example, the penetrating member may be provided with a groove or indentation along its length or, when viewed in cross-section, it may comprise a partial circle.

As shown in FIGS. 1 and 2, the central lumen 13 of catheter 12 houses a stainless steel or Nitinol hollow penetrating member 20. The penetrating member 20 is provided with a sharp tip 22 at its distal end, which is used to puncture and penetrate tissue at the site of treatment. The lumen of the penetrating member 20 allows for venting of gas and fluid that is liberated during tissue ablation. Substances, including drugs, may be administered to the site of treatment through the lumen of the penetrating member 20. Furthermore, the lumen may be used to deliver devices such as nanosensors to the site of treatment. The lumen of the penetrating member 20 may also be used as an aspiration channel to extract fluids from the site of treatment and/or to take a tissue biopsy. A tissue biopsy can be taken to determine the presence or extent of a disease at the site of treatment, e.g. a malignant tumour. Furthermore, the lumen of the penetrating member can act as a guidewire channel. In embodiments of the invention where the penetrating member 20 does not comprise a lumen, equivalent benefits can be achieved via the aforementioned optional longitudinal groove or channel formed along its length.

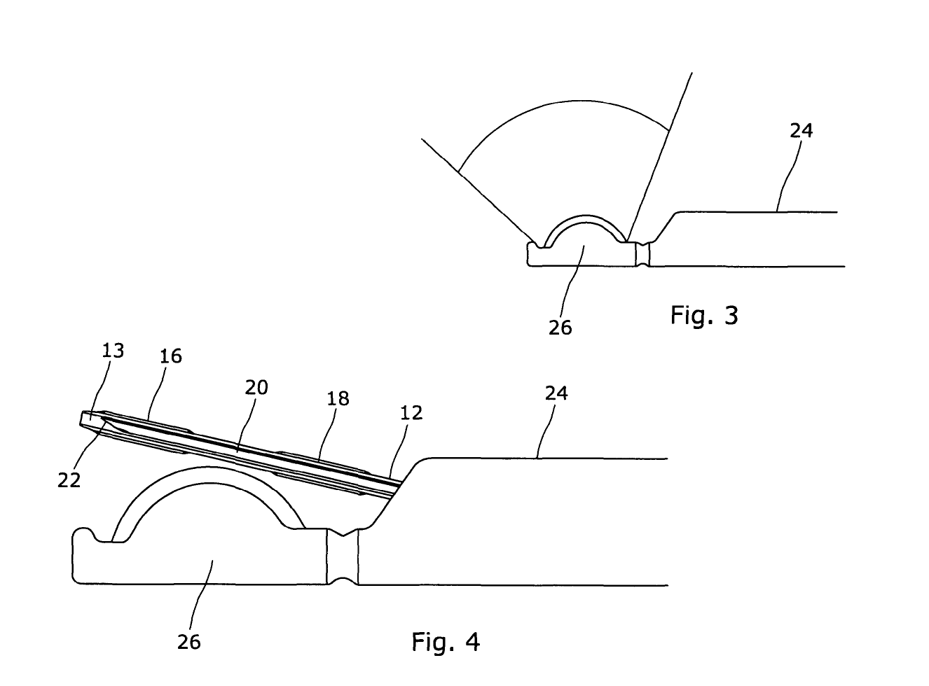

In an embodiment of the invention, the catheter 12 can be delivered towards the site of treatment through the lumen of an endoscope 24. In this configuration, the endoscope 24, the catheter 12 and the penetrating member 20 are coaxially aligned with one another. In use, the proximal ends of the endoscope 24, the catheter 12 and the penetrating member 20 are located on the outside of the body of the patient so as to permit control by the user. The distal ends are advanced towards and ultimately located near or at the site of treatment within the body. As shown in FIG. 3, the endoscope 24 may be provided with an ultrasound transducer 26 at its distal end to provide an image of the site of treatment. This allows precise visualisation and positioning of the distal end of the catheter and/or penetrating member so that ablation energy can be administered accurately to the targeted tissue at the site of treatment. The distal end of the endoscope includes the distal tip (which is synonymous with the distal terminus of the endoscope) and the area close to or adjacent to the distal tip.

In an embodiment of the invention, the device extends the effective range of the endoscope once the endoscope has reached its maximum length of deployment. It is also possible for the device to be extended beyond the field of view of the ultrasound endoscope.

Typically, the apparatus of the invention are operated according to three main phases of therapy: an insertion phase, a therapy phase and a removal phase. The insertion phase includes the endoscopic insertion of the device (optionally preceded by the insertion of a guidewire through the lumen of the penetrating member, if required) and the location of the device to the site of treatment where therapy is to be administered. The therapy phase includes administering sufficient energy to thermally ablate the surrounding tissue. The removal phase includes the withdrawal of the device from the site of treatment, usually back along the initial insertion route.

In the insertion phase, the endoscope 24 housing the catheter 12 is advanced along the desired hollow organ—for example, the gastrointestinal tract, including the esophagus, the stomach and duodenum, the small intestine, the large intestine\colon, the bile duct and the rectum; the respiratory tract; the urinary tract; or the female reproductive system, including the uterus and the fallopian tubes—towards the site of treatment. During the insertion phase, the penetrating member 20 is retained completely within the lumen 13 of catheter 12 as shown in FIG. 4. This is referred to as the retracted position and prevents the sharp tip 22 of the penetrating member 20 from damaging the inner surface of the endoscope 24 or the ultrasound transducer 26. When in the retracted position, the penetrating member 20 provides additional structural support to the catheter 12.

It is also possible for the endoscope 24 to be inserted into the body and for the catheter 12 to be inserted into the lumen of the endoscope only once the distal end of the endoscope has reached the desired location.

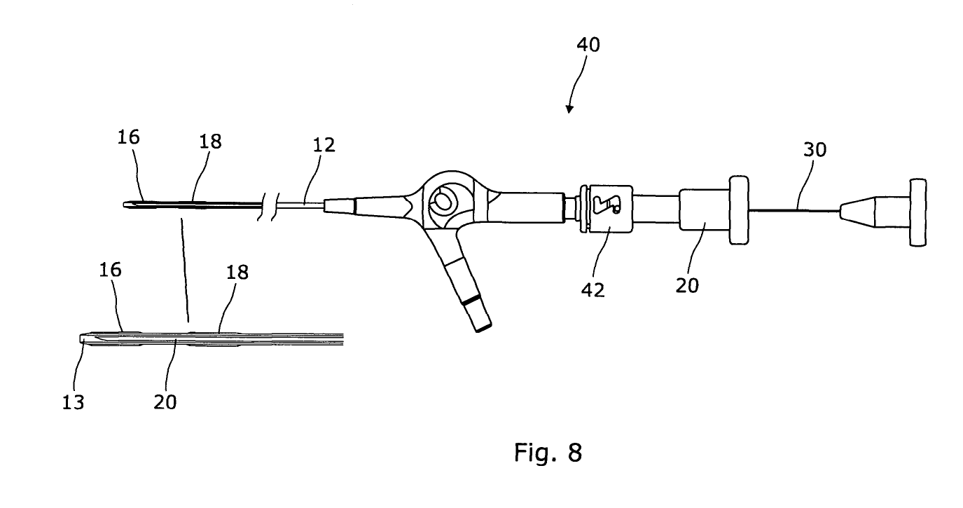

As shown in FIG. 7, advancement and retraction of the catheter 12 and penetrating member 20 into and out of the site of treatment is controlled by a hub 40 located at the proximal end of the catheter 12, which is located outside of the body of the patient when in use. The hub comprises a twist lock 42, which retains the penetrating member 20 in coaxial alignment within the lumen 13 of the catheter 12. Once the penetrating member 20 is locked in position within the lumen 13 of the catheter 12, the device can be loaded into the lumen of the endoscope 24. By rotating the twist lock 42, the user can advance or retract the penetrating member 20 within the lumen 13 of the catheter 12 in a controlled manner at will. The twist lock may have settings for coarse and fine advancement of the penetrating member. The length of the twist lock will determine the length of exposure of the penetrating member 20 beyond the distal end of the catheter 12. Typically the catheter 12 and/or the penetrating member 20 will be advanced between 1 mm and 200 mm, preferably between 5 mm and 100 mm, more preferably between 10 mm and 50 mm.

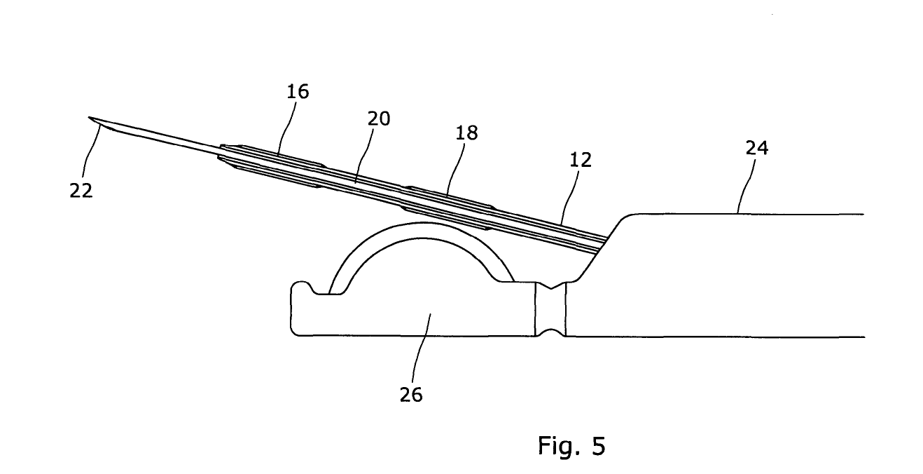

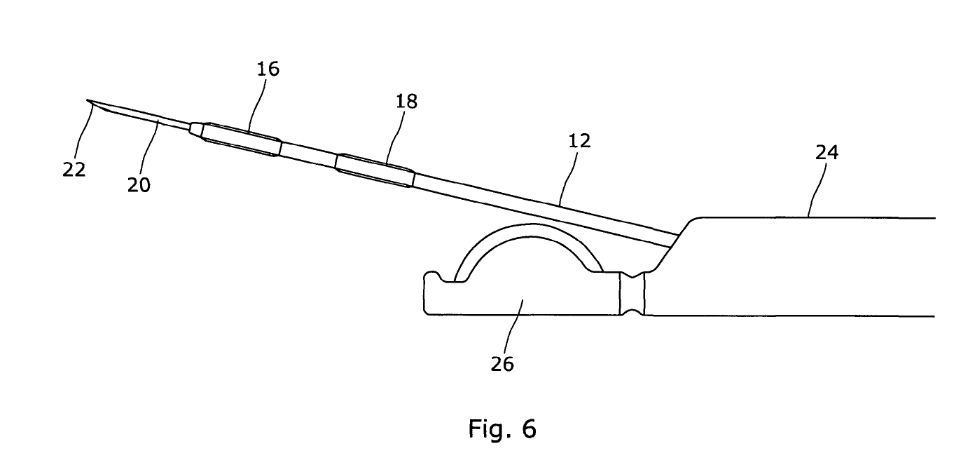

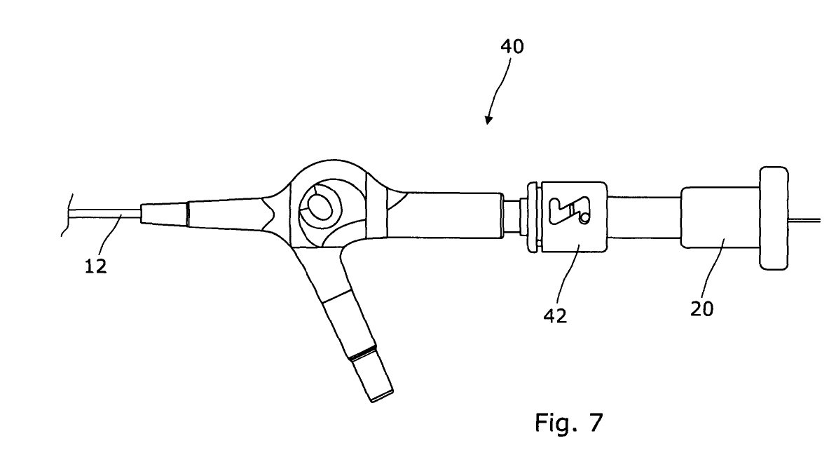

When the distal end of the endoscope 24 is positioned adjacent to or in proximity of the site of treatment, the distal end of catheter 12 is advanced out of the lumen of the endoscope 24. Once the distal end of the catheter 12 has been advanced beyond the ultrasound transducer 26, the distal end of the penetrating member 20 is advanced out of the lumen 13 of the catheter 12, as shown in FIG. 5, and the sharp tip 22 of the penetrating member 20 penetrates the tissue wall of the hollow organ in which the endoscope is located. The distal end of the catheter 12 is then advanced into the tissue through the puncture hole/wound/tract created by the penetrating member 20, as shown in FIG. 6, until the distal electrodes 16 and 18 are located at or adjacent to the desired site of treatment. The site of treatment is typically a lesion or a tumour.

During the therapy phase, RF current is activated so that a controlled heating zone is formed between the distal 16 and proximal 18 electrodes (see FIG. 14(a)). The RF current is suitably at a frequency between 100 kHz and 5 MHz, preferably a frequency of around 460 kHz. The time of activation is typically between about 0.1 seconds and about 180 seconds. This causes ablation of surrounding tissue at the site of treatment. Where the device is to be used with an endoscope having an ultrasonic transducer at its distal end, it is typical to use low heat for a short amount of time in order to avoid possible damage to the ultrasonic transducer. The ultrasound transducer 26 is used to locate the primary tissue target within the field of imaging and to position the distal end of the catheter 12 at the desired site of treatment. Ultrasound waves emitted from the transducer 26 are reflected off the surface of the distal end of the penetrating member 20 and the distal end of the catheter 12, as well as the surrounding tissue, and are returned to the transducer to provide an image of the site of treatment. The heating zone created by the bipolar electrode arrangement causes fluid in the surrounding tissue to reach boiling temperature and the gas released further assists in ultrasound imaging of the site of treatment.

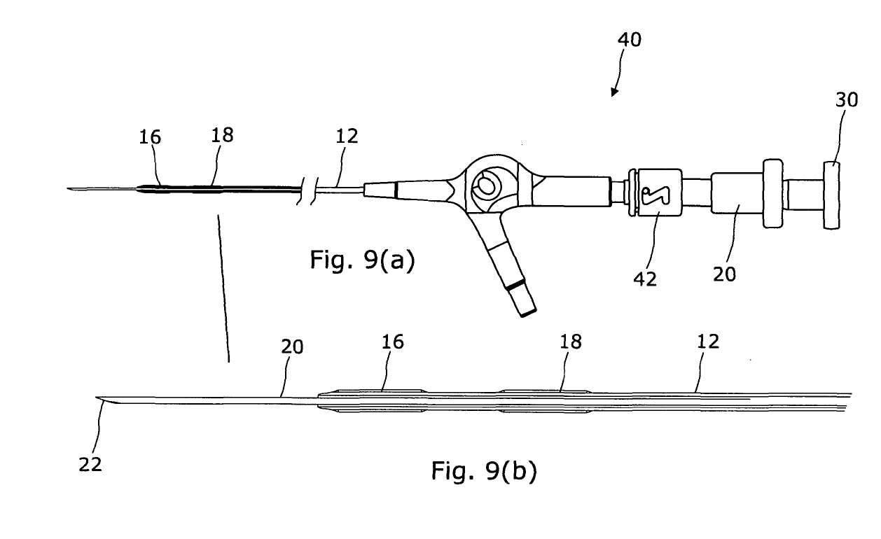

The penetrating member 20 may be retracted into the catheter 12 prior to ablation or may remain in the extended configuration throughout ablation as shown in FIG. 6. In the situation where a guidewire 30 is located in the lumen of the penetrating member 20 (as shown in FIGS. 8 to 10), retaining the penetrating member 20 in the extended configuration throughout ablation has the advantage of protecting the guidewire 30 from possible heat damage.

Once ablation of the primary tissue target is complete, the catheter 12 together with the penetrating member 20 is withdrawn from the site of treatment into the lumen of the endoscope 24 and the endoscope 24 can be withdrawn from the hollow organ. Alternatively, the device can be deployed to another location nearby.

An issue that can potentially arise during treatment is sticking/adherence of one or more of the electrodes of the device to the tissue that is being ablated at the treatment site. Clearly this is undesirable in instances where the treatment site is close to critical organs, nerves or blood vessels and where withdrawal of the device would lead to additional tissue trauma due to tearing. In a specific embodiment of the invention, one way of reducing or avoiding tissue stick/adherence is to continually or intermittently rotate and/or advance the device back and forth slightly (for example by a few millimetres) during treatment. Rotation and/or lateral movement of the device may be controlled externally of the patient at the proximal end of the device by the user either manually or automatically using a rotation/lateral movement device. Continual or intermittent movement of the device helps to reduce the likelihood of the electrodes adhering to the tissue.

Optionally, the distal electrodes 16 and 18 of the device can be used to cauterise and seal the puncture wound as the device is withdrawn from the site of treatment. The puncture wound can also be sealed by others means known to the skilled person such as by stitching or stapling. Any drugs and/or devices which have been delivered to the site of treatment though the lumen of the penetrating member 20 can be trapped in the site of treatment by sealing the puncture would.

In an alternative embodiment of the invention, instead of being delivered endoscopically to the site of treatment, the device can be inserted percutaneously through an incision made in the skin of the patient. The device can be guided to the site of treatment via the circulatory system of arteries and veins.

In yet another embodiment of the invention (not shown), the device can be used to induce endoluminal closure of hollow anatomical structures such as blood vessels of a range of diameters from large to small.

In another embodiment of the invention, the device can be delivered directly to the site of ablation through a natural orifice into a hollow organ, vessel or cavity of the body, e.g. the GI tract, without the use of an endoscope.

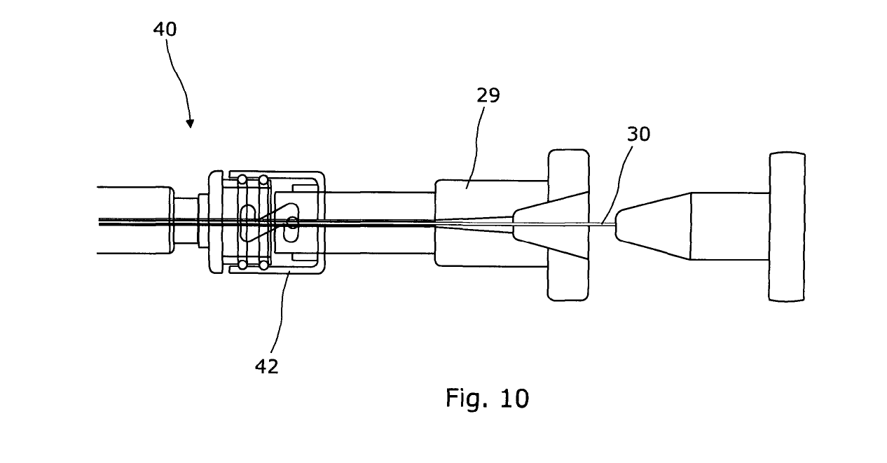

As shown in FIGS. 8 to 10, in an alternative embodiment of the invention, a guidewire 30 may be housed within the lumen of the penetrating member 20 to assist with tracking of the device. The guidewire 30 can be removed from the penetrating member 20 once the penetrating member 20 and the catheter 12 have been positioned at the desired site of treatment. As shown most clearly in FIG. 10, the proximal end of the guidewire 30 can be at least partially accommodated within the proximal end of the penetrating member 20. Alternatively, the guidewire may be left in position after removal of the device to allow other devices to access the therapy location.

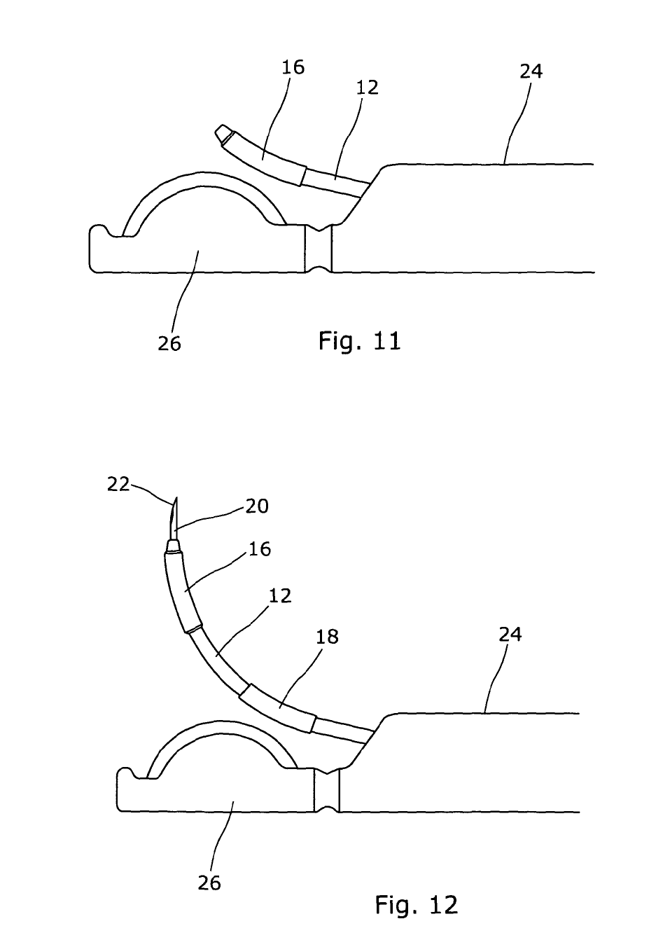

Another embodiment of the invention is shown in FIGS. 11 to 13. The device is similar to the device of the first embodiment of the invention except that the catheter 12 can be made to curve as it is advanced out of the distal end of the endoscope 24 by using a heat formed sprung wire or Nitinol comprised within the catheter body. The degree of curvature will depend in part upon the distance between the exit point of the catheter 12 from the endoscope 24 and the tissue entry point. The curving of the catheter 12 advantageously allows the device to follow a track within the field of imaging of the ultrasound transducer 26. It also enables the device to be used in areas where the local anatomy of the patient is particularly challenging.

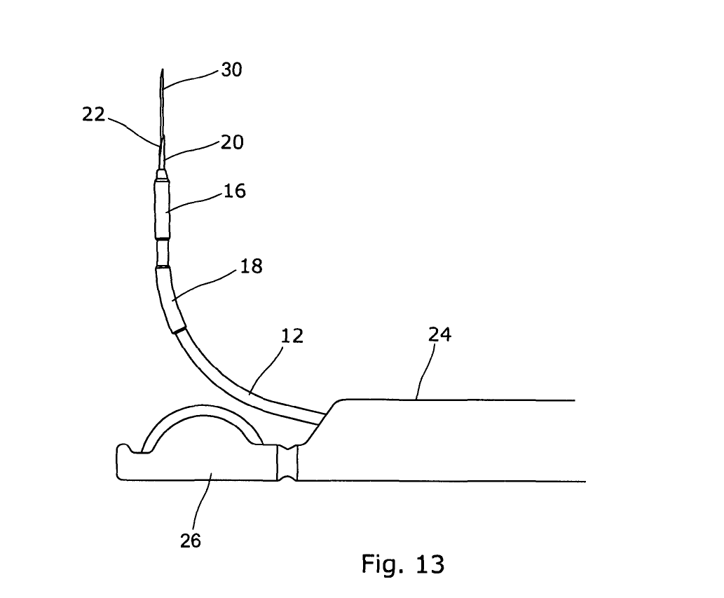

As shown in FIG. 13, in an embodiment of the invention, it is possible for a guidewire 30 to be housed within the lumen of the penetrating member 20.

In another embodiment of the invention, the catheter 12 is provided with a bipolar RF electrode arrangement at its distal end as previously described and, in addition, the penetrating member 20 is also provided with an RF electrode 17 at its distal end. The distal end of the penetrating member includes the distal tip (which is synonymous with the distal terminus of the penetrating member) and the area close to or adjacent to the distal tip. As shown in FIG. 14(b), the addition of an electrode 17 on the penetrating member 20 increases the effective heating field of the device thereby lengthening the ablation zone. The electrode 17 can also advantageously be used to seal and close the site of puncture of the hollow organ wall as the device is withdrawn from the body after treatment. By increasing the distance between the electrode 17 and the distal tip of the catheter 12, the shape and length of the ablation zone can be varied as required by the user. Penetrating members comprising a bipolar RF electrode arrangement or an array of RF electrodes may also be used.

In an alternative embodiment of the invention (not shown), the device is provided with a single radiofrequency (RE) electrode (a monopolar electrode arrangement) at its distal end. A grounding pad in contact with the patient's body provides the other electrode polarity and completes the RF circuit. The monopolar electrode and the grounding pad are connected to opposite polarities of an RF energy source. When the device is in use, RF current flows between the monopolar electrode and the grounding pad, resulting in a local heating zone around the monopolar electrode, which is used to ablate abnormal tissue at the site of treatment.

In another embodiment of the invention (not shown), the device may comprise an array of electrodes so that thermal ablation can take place along an increased proportion of the site of treatment.

In alternative embodiments of the invention (not shown), microwave energy, ultrasound energy, irreversible electroporation and an electric current are used to apply energy to the site of treatment, either in addition to or instead of RF energy. In the case of microwave energy, two conducting cylinders can be mounted on the elongate body of the device with a small interval between them such that they form a dipole antenna. The cylinders are connected to a coaxial cable which can be supplied with microwave energy at frequencies between 200 MHz and 5 GHz. When microwave energy is applied to the coaxial cable the dipole will act as a source of microwave radiation, which will propagate as a cylindrical wave, depositing heat in the region next to the device.

In the case of ultrasound energy, a cylinder of a piezoelectric material such as PZT-4 can be mounted on the distal end of the device. Electrodes, suitably made from silver, gold, or a titanium or tungsten alloy, are typically plated on the inner and outer surface of the cylinder. RF energy can be applied between the electrodes at an ultrasound frequency, for example the energy will typically be between 200 kHz and 20 MHz. This generates a cylindrical ultrasound wave which will radiate outwards and cause tissue ablation.

In the case of irreversible electroporation (IRE), a rapidly pulsing electric field is generated within an electrode arrangement thereby creating permanent pores in the membrane of the surrounding tissue cells. Damage to the cell membrane causes cell death through the loss of cell homeostasis in a non-thermal manner. IRE results in a highly focussed and well defined ablation zone and can reduce peripheral damage to healthy tissue, blood vessels and connective tissue.

In a specific embodiment of the invention, a bipolar (or multipolar) arrangement is provided at the distal end of the device whereby the energy delivery elements comprise electrodes capable of delivering a high electric field in micro to nano-second pulses. The electrodes are in contact with an IRE generator located outside of the body of the patient (e.g. NanoKnife® IRE System, AngioDynamics, Inc., Queensbury N.Y., USA; or Cliniporator™, Igea, Carpi, Italy) and can deliver a direct current electrical field up to around 3 kV in a plurality of pulses ranging from nanoseconds up to around 100 microseconds in length. Typically, at least 2 and at most 500 pulses are administered per lesion, dependent upon the size of the tissue to be ablated. Electrode design and placement for use in IRE embodiments of the present invention are substantially the same as for RF embodiments described herein.

In the case of electric current energy, aside from radiofrequency ablation, the electric current can take the form of resistive heating.

Penetrating members comprising a monopolar RF electrode arrangement, a bipolar RF electrode arrangement or an array of RF electrodes may be used with any of the afore-mentioned devices having various different energy delivery sources/elements, e.g. one or more RF electrodes, microwave energy, ultrasound energy, irreversible electroporation, electric current, etc. Furthermore, the penetrating member itself may be provided with any of the afore-mentioned alternative energy delivery elements e.g. microwave energy, ultrasound energy, irreversible electroporation, electric current in the form of resistive heating, etc, instead of or in addition to one or more RF electrodes. For example, in the case where the device is provided with a single radiofrequency (RF) electrode (a monopolar electrode arrangement) at its distal end, a penetrating member comprising a single RF electrode, preferably at its distal end, may be used instead of a grounding pad to complete the RF circuit.

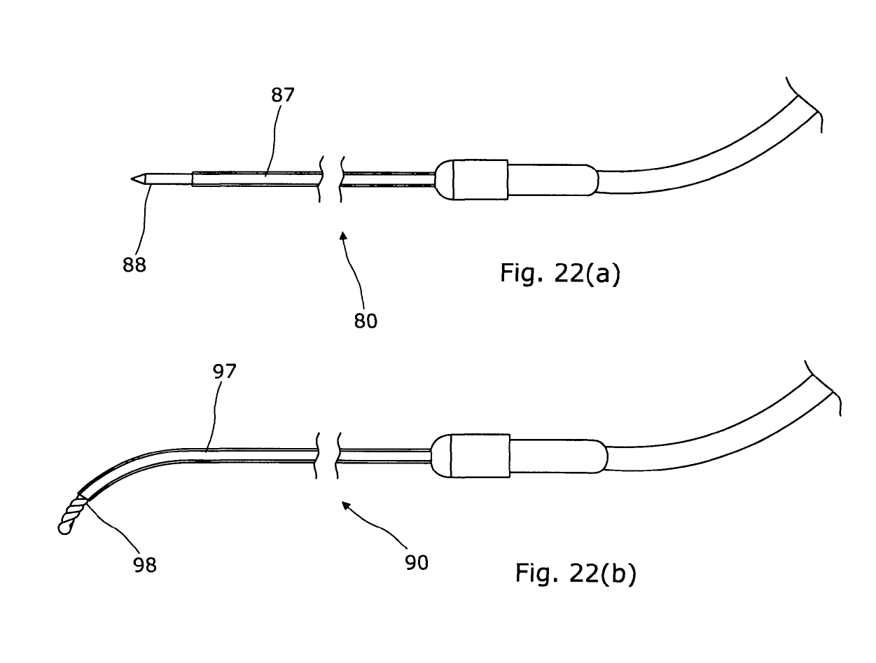

An alternative embodiment of the invention is shown in FIGS. 22-24. The ablation device (also referred to as the device) comprises an elongate body including a conductive core member extending along its length and an outer sleeve of an insulating or non-conductive material. The ablation device typically comprises a catheter of small diameter (less than 0.6 mm) or a guidewire. FIG. 22 (a) shows an ablation device 80 in which the central conductive core region is exposed at the distal tip to form an uncoated electrode 88. The sleeve or coating 87 serves to insulate the remainder of the device from the surroundings so as to prevent ablation or short circuiting outside of a controlled zone. The electrode 88 shown in FIG. 22(a) comprises a tapered or pointed tip in order to facilitate tissue penetration, although in alternative embodiments the tip may be blunt ended. The central core of the device 80 comprises a conductive material such a metal or metal alloy, including steel, Nitinol, gold or platinum, thereby allowing connection to an energy generator such as an RF generator or irreversible electroporation generator, located externally.

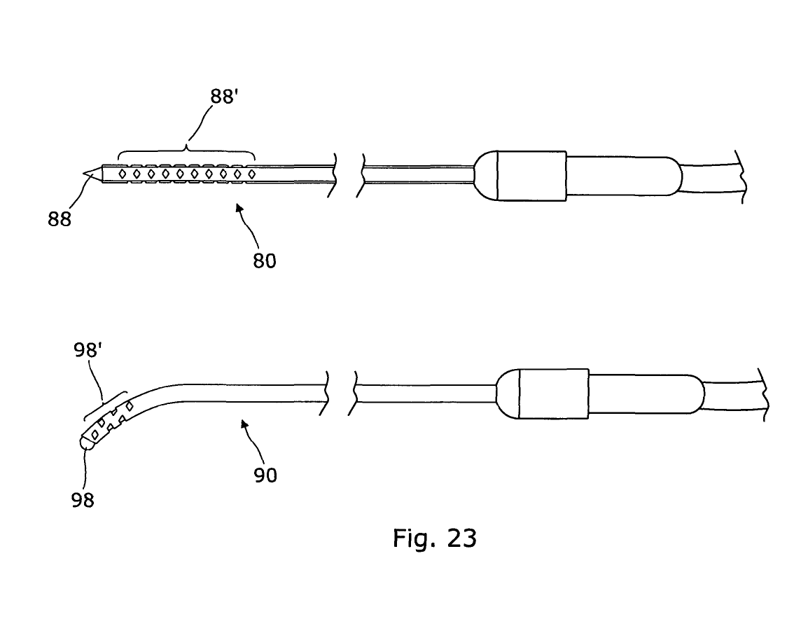

In FIG. 22(b) the device 90 comprises a coiled or braided conducting core that is exposed at the distal tip to form an electrode 98. Use of a coiled conducting core provides the advantage of increasing the flexibility of the device in use. It is optional to modify the electrodes of the devices 80, 90 by providing one or more windows 88′, 98′ formed by introducing apertures into the outer insulating coating 87, 97 (see FIG. 23). The effect of the windows 88′, 98′ is two-fold. Firstly, to increase the ultrasound echogenicity of the tip of the ablation device 80, 90 (see later discussion on ultrasound echogenicity), and secondly to control the power distribution and energy delivery characteristics of the electrode 88, 98.

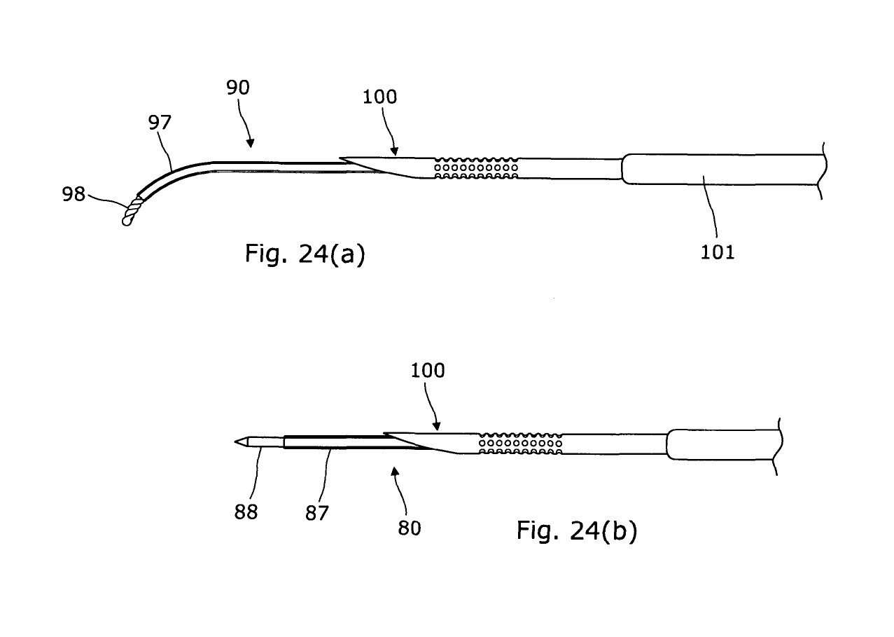

The ablation device 80, 90 is suited to coaxial placement within the central lumen of a piercing or penetrating member 100 (see FIGS. 24(a) and (b)) having a sharpened or pointed distal end. In an embodiment of the invention in use, the penetrating member 100 is located within a lumen of an endoscope 24 (suitably within a biopsy channel), and the endoscope is placed within the body of a patient at a position adjacent to a site requiring treatment, such as the site of a lesion or tumour. The penetrating member may comprise the ablation device 80, 90 within a central lumen. Typically, the penetrating member 100 will be advanced distally from the endoscope 24 (optionally under ultrasound or other guidance) into the tissue until the distal tip of the penetrating member 100 is located within or sufficiently close to the lesion. At this point the ablation device 80, 90 may be advanced from the distal tip of the penetrating member 100 and energy applied to the tissue or lesion one or more times as necessary. On completion of the ablation phase the device 80, 90 can be withdrawn into the penetrating member 100 which in turn can be withdrawn from the tissue back into the central lumen of the endoscope 24.

In alternative arrangements (not shown), instead of having a single exposed region of conductive core forming a monopolar electrode at the distal tip, the ablation device (e.g. a narrow catheter or a guidewire) may comprise two or more exposed regions to form a bipolar electrode arrangement or an array of electrodes. The most distal electrode may be located at or close to the distal tip of the device. The electrodes may confer RF energy, resistive heating energy or irreversible electroporation energy to the site of treatment. Furthermore, the penetrating member may be provided with one or more energy delivery elements e.g. RF energy, microwave energy, ultrasound energy, irreversible electroporation, electric current in the form of resistive heating, etc, as discussed in relation to any of the previous embodiments.

Instead of the ablation device having uncoated electrodes as described above and as shown in FIGS. 22-24, i.e. formed by exposing the central conductive core of the device, the electrodes may be formed by any other method or take any other form known in the art. For example, the electrodes may be manufactured separately from the conductive core and may be attached to the device so that they are in connection with the conductive core or are otherwise connected to an externally located energy generator. The electrodes may confer RF energy, resistive heating energy or irreversible electroporation energy to the site of treatment. Alternatively, the ablation device may comprise one or more energy delivery elements capable of delivering microwave energy or ultrasound energy, as discussed previously.

In an alternative embodiment of the invention the ablation device 80, 90 (as shown in any of FIGS. 22-24 or as described in any of the alternative embodiments above, e.g. a monopolar or bipolar narrow catheter or guidewire, etc.) can also be used in combination with a catheter in place of the penetrating member, i.e. the ablation device may be located within a lumen of a catheter. The catheter may take the form of the catheter as shown in any of FIGS. 1-21 or as described in any of the embodiments above, e.g. a monopolar or bipolar catheter having one or more energy delivery sources/elements, such as RF electrodes, IRE, etc. For example, a monopolar ablation device can be used with a monopolar catheter to create a bipolar circuit, which may be a bipolar RF energy circuit. Optionally, the catheter housing the ablation device in its lumen may be located within a lumen of an endoscope (suitably within a biopsy channel), and the endoscope may be placed within the body of a patient at a position adjacent to a site requiring treatment, such as the site of a lesion or tumour. In the case where the ablation device comprises a distal electrode with a pointed or sharpened tip (as shown, for example, in FIG. 22(a)), the electrode itself may be used to pierce tissue create a channel for the device and catheter to access the site of treatment. In the case where the electrode is blunt-ended, a penetrating or piercing member or probe (in any of the forms described above) may first be advanced along the lumen of the endoscope to pierce the tissue, and optionally take a tissue biopsy, and then retracted and removed from the endoscope. After removal of the penetrating member from the endoscope, the ablation device and the coaxially aligned outer catheter may be advanced down the lumen of the endoscope towards the site of treatment, either simultaneously or consecutively.

In a further embodiment of the invention (not shown) the ablation device (according to any of the embodiments described above, e.g. monopolar or bipolar catheter, narrow catheter or guidewire, etc) may be located within a lumen of an endoscope (suitably within a biopsy channel). In this arrangement in use, there is no requirement for a coaxially aligned catheter or a coaxially aligned penetrating member. In the case where the ablation device comprises a distal electrode with a pointed or sharpened tip (as shown, for example, in FIG. 22(a)), the electrode itself may be used to pierce tissue create a channel for the device to access the site of treatment. In the case where the electrode is blunt-ended, a piercing or penetrating member or probe (according to any of the embodiments described above) may first be advanced along the lumen of the endoscope to pierce the tissue and then removed from the endoscope. After removal of penetrating member, the ablation device may be advanced down the lumen of the endoscope towards the site of treatment.

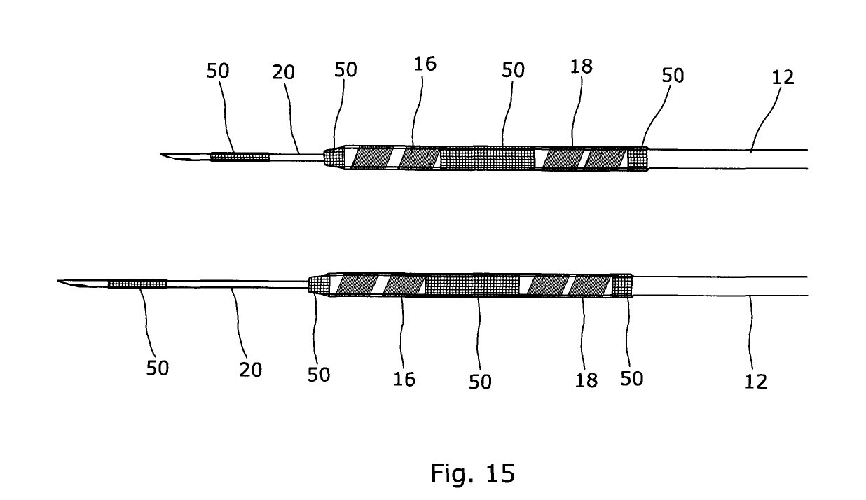

In another embodiment of the invention, the device includes any of the previous embodiments but further comprises enhanced echogenic surfaces to improve ultrasound imaging and positioning of the device at the desired site of treatment. Enhanced ultrasound echogenicity is provided by one or more areas or portions of increased ultrasound reflection on the surface of the device, particularly the surface of the distal end of the device, and optionally on the surface of the penetrating member (according to any of the embodiments described above) and/or the surface of the guidewire 30. As shown in FIG. 15, the areas of increased ultrasound reflection 50 may be located on the surface of the device (catheter 12) adjacent to and between the bipolar electrodes 16 and 18, as well as on the surface of the penetrating member 20.

As used herein, the term ‘ultrasound reflection’ includes both specular and scattered reflected ultrasound waves. Specularly reflected waves are typically regarded as those which are bounced back from a surface at an angle which mirrors the angle of incidence and do not return to the transducer unless the surface is perpendicular to the ultrasound wave. Scattered waves reflect at a wide range of angles and a fraction of these waves will be returned to the ultrasound transducer.

In previous embodiments of the invention the device (e.g. catheter 12 or narrow catheter/guidewire 80, 90), the energy delivery elements (e.g. electrodes 16 and 18, 88 and 98), the penetrating member (e.g. 20 and 100) and the guidewire 30 are all generally cylindrical or annular in shape with smooth surfaces. This means that many of the incident ultrasound waves striking these surfaces are specularly reflected in a direction away from the ultrasound transducer 26 and the echo signal returning to the ultrasound transducer 26 can thus be relatively weak. This can lead to an imprecise image of the site of treatment. By placing areas of increased ultrasound reflection on the surface of the device, the surface of the penetrating member and/or the surface of the guidewire 30, the incident ultrasound waves striking these surfaces are reflected in many different directions and ultimately more of the returning ultrasound waves are reflected towards the ultrasound transducer 26. This means that the echo signal returning to the ultrasound transducer 26 is generally stronger and results in an improved and more detailed image of the site of treatment, which enables the electrodes or energy delivery elements, for example electrodes 16 and 18, or 88 or 98, to be positioned more accurately for cautery treatment of the lesion.

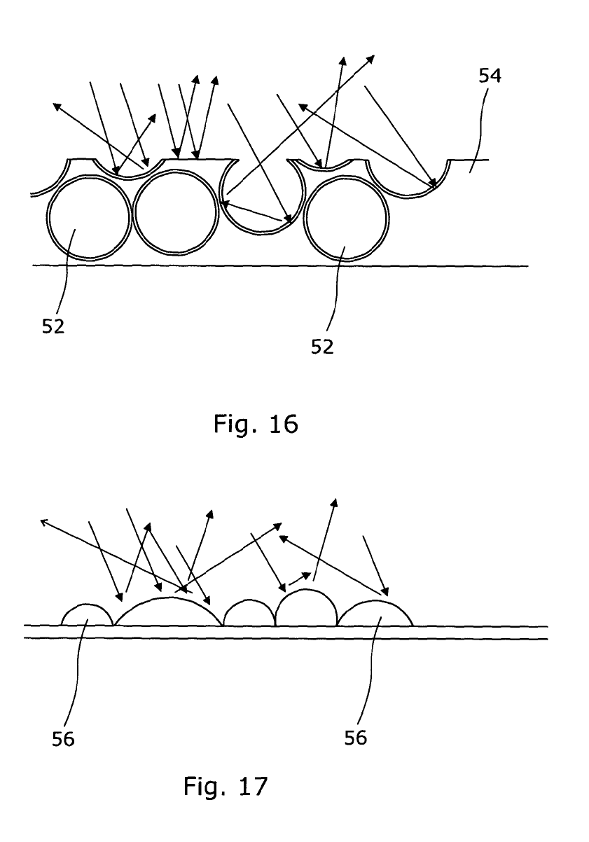

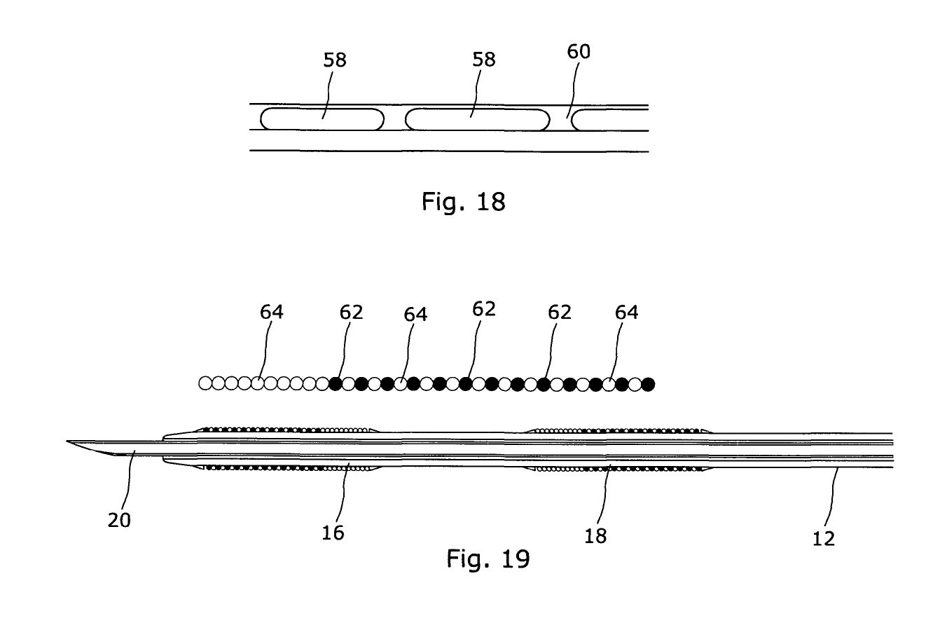

There are various different ways of increasing ultrasound echo, scatter and reflection. For example, as shown in FIG. 16, a fine coating layer of glass or polymer gas filled micro-balloons or bubbles 52 in an adhesive or polymer matrix 54 can be applied to the surface of the device, the penetrating member and/or guidewire 30. The micro balloons and gas pockets of the coating provide structured aeration of the surface and enhance surface scatter. Alternatively, as shown in FIG. 17, polymers or metals can be sputtered onto the surface of the device, the penetrating member and/or guidewire 30 to form uneven surface globules 56 which scatter the ultrasound waves. As shown in FIG. 18, gas pockets 58 may be trapped in a silicone or polymer coating 60 to enhance ultrasound reflection. Various biocompatible polymers can be used for the coating, including polyurethane, structured hydrogels, polyether block amide (PEBA), expanded polytetrafluoroethylene (ePTFE) and poly(p-xylylene) polymers.

Areas of increased ultrasound reflection 50 may also be located on the surface of the one or more energy delivery elements, for example the bipolar electrodes 16 and 18, instead of or in addition to the areas of increased ultrasound reflection on the surface of the device adjacent to and between the energy delivery elements. For example, as shown in FIG. 19, a hollow micro fibre 62 can be interspersed between the conductor 64 of the electrodes 16, 18 to improve ultrasound imaging. The hollow micro fibre 62 and conductor 64 form a double spiral around the electrodes and air trapped in the hollow micro fibre 62 increases ultrasound echo.

Other ways in which the surfaces of the device can be modified to increase ultrasound echo include providing the surfaces with a plurality of recesses and/or projections, a plurality of grooves and/or ridges, or a combination thereof. A roughened surface can also be created by using an abrasive, such as by microblasting the surface of the device with particles or beads.

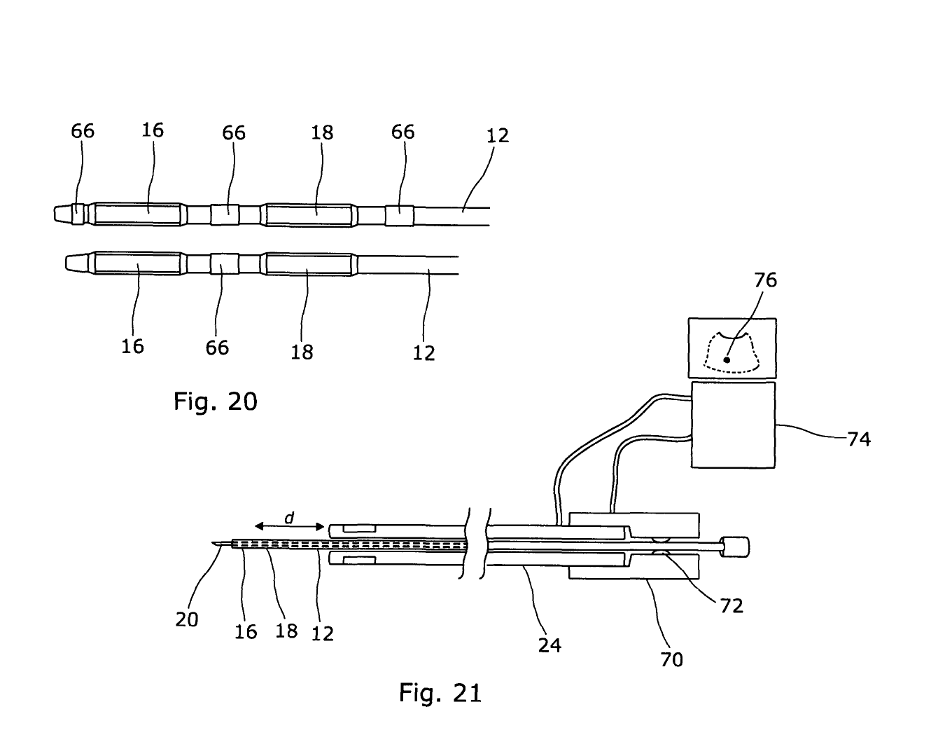

Increased ultrasound echo can also be provided by mounting one or more ultrasound transmitting elements on the ablation device. A piezoelectric material such as PZT (lead zirconate titanate) or PVDF (polyvinylidene fluoride) 66 can be mounted adjacent to and/or between the energy delivery elements, such as the bipolar electrodes 16 and 18 as shown in FIG. 20, and excited with a signal synchronised with the ultrasonic endoscope 24. Single or multiple PZT or PVDF elements 66 can be used depending upon the range of the ultrasound endoscope. The elements can take the form of a ring, crystal or film of piezoelectric material. The signal generated by the PZT or PVDF elements 66 is detectable by the ultrasonic endoscope 24. PZT or PVDF elements 66 can be used in conjunction with the surface modifications discussed above to improve ultrasound echo.

In another embodiment of the invention (not shown), a non-ultrasonic endoscope is used to deliver the device to the site of treatment instead of an ultrasonic endoscope. In this instance, in order to image and position the device at the site of treatment, an external ultrasound transducer is used. The external ultrasound transducer is moved across the appropriate area of body of the patient in order to visualise the site of treatment. Ultrasound waves are emitted from the external transducer and penetrate through the body tissue towards the site of treatment. Incident ultrasound waves are reflected from the surface of the device and are detected by the external ultrasound transducer. As described above, one or more areas of increased ultrasound reflection (echogenic surfaces) can be provided on the surface of the ablation device, and/or the surface of penetrating member and/or the surface of the guidewire 30 to increase the ultrasound reflection and improve visualisation of the device at the site of treatment.

In yet another embodiment of the invention (not shown), a non-ultrasonic endoscope is used to deliver the device to the site of treatment instead of an ultrasonic endoscope, and the ablation device is provided with an ultrasound transmitter at its distal end to assist with navigation. Typically, the ultrasound transmitter is located proximally to the one or more energy delivery elements at the distal end of the device, for example proximally to the bipolar electrodes 16 and 18 at the distal end of the catheter 12. The ultrasound signal may be received by an external ultrasound receiver/sensor located on the surface of the body of the patient. Ultrasound waves emitted from the internal ultrasound transmitter are reflected off the surface of the device and are detected by the external ultrasound transducer. As described above, one or more areas of increased ultrasound reflection (echogenic surfaces) can be provided on the surface of the device, and/or the surface of penetrating member and/or the surface of the guidewire 30 to increase the ultrasound echo and improve visualisation of the device at the site of treatment.

In another embodiment of the invention (not shown) a bipolar catheter is provided with an ultrasound transmitter at its distal end to assist with navigation. A non-ultrasonic endoscope may be used to deliver the catheter to the site of treatment. Alternatively, the catheter may be inserted percutaneously through an incision made in the skin of the patient, or it can be guided to the site of treatment via the circulatory system of arteries and veins, or it can be delivered directly to the site of treatment through a natural orifice into a hollow organ, vessel or cavity of the body, e.g. the GI tract, without the use of an endoscope. Typically, the ultrasound transmitter is located proximally to the bipolar electrodes at the distal end of the catheter. The ultrasound signal may be received by an external ultrasound receiver/sensor located on the surface of the body of the patient. Ultrasound waves emitted from the internal ultrasound transmitter are reflected off the surface of the device and are detected by the external ultrasound transducer. As described above, one or more areas of increased ultrasound reflection (echogenic surfaces) can be provided on the surface of the catheter to increase the ultrasound echo and improve visualisation of the catheter at the site of treatment.

In an embodiment of the invention (not shown), a microwave or electromagnetic transmitter is located at or close to the distal tip of the device and is used to assist with navigation of the device under ultrasound scan, CT or MRI. The transmitter may be an electromagnetic coil that can be received by a set of external reference coils, such as the Flock of Birds system (Ascension Technologies, Burlington, Vt.) to give a three-dimensional 3D position. Alternatively, the electromagnetic transmitter may comprise an MR tracking coil (C. L. Dumoulin, ‘Active Visualisation MR-Tracking’, pages 65-75, Interventional Magnetic Resonance Imaging, Springer-Verlag, Berlin, Germany 1998; and C. L. Dumoulin, et al. ‘Tracking system to follow the position and orientation of a device with radiofrequency field gradients’, Technical report U.S. Pat. No. 5,211,165, USPTO, Department of Commerce, Arlington, Va., USA, 1993).

In another embodiment of the invention (not shown), a microwave or electromagnetic transmitter is located at or close to the distal tip of a bipolar catheter and is used to assist with navigation of the catheter under ultrasound scan, CT or MRI. As above, the transmitter may be an electromagnetic coil that can be received by a set of external reference coils, such as the Flock of Birds system (Ascension Technologies, Burlington, Vt.) to give a 3D position. Alternatively, the electromagnetic transmitter may be an MR tracking coil.

In another embodiment of the invention as shown in FIG. 21, the proximal end of the device (in this case catheter 12) can be passed through a motion detector 70 located at the proximal end of the ultrasonic endoscope 24. The motion detector 70 may have a wheel 72 connected to a potentiometer or an optical motion connector. The motion detector 70 permits measurement of the extent ‘d’ that the catheter 12 protrudes from the distal tip of the endoscope 24. This measurement can be fed into an ultrasound scanner 74 so that the position of the catheter tip can be superimposed on the ultrasound image 76.

The device, the penetrating member and/or the guidewire 30 of any embodiment of the invention can be provided with marker bands to allow estimation of the depth of tissue penetration by the device, the penetrating member and/or the guidewire 30. The marker bands can be formed from a high-density material or radio-opaque material so that they can be visualised. Suitable radio-opaque materials include gold, platinum, etc, or polymers doped with a radio-opaque material. A radio-opaque material, such as a platinum or titanium band, can also be placed on the tip of the penetrating member and/or the distal tip of the device so that the deployment distance can be visualised. Printed marker bands can also be provided on the elongate body of the device towards the proximal end of the device so that the user can see from the portion of the device located externally of the body of the patient how far the device has been advanced. In one embodiment of the invention the proximal terminus of the, ablation device, penetrating member and/or the guidewire 30 can be located within a slider housing in order to facilitate fine control of deployment.

To enhance visualisation of the device under MRI, gadolinium can be incorporated into the device, for example in the form of a coiled wire on the surface of the ablation device or in the form of marker bands to allow estimation of the depth of tissue penetration by the device. Gadolinium can also be used in this manner on a bipolar electrode arrangement catheter to enhance visualisation of the catheter under MRI.

It is possible to monitor the progress of the therapy phase by including at least one temperature sensor (not shown), such as a thermocouple, on the device of the invention. Typically, the temperature sensor is provided at the distal end of the device, typically either between the energy delivery elements, e.g. electrodes, or at or close to the distal tip of the device.

In all embodiments of the invention, the device body is suitably manufactured from plastics or polymeric biocompatible materials known in the technical field, e.g. PTFE or PET. The device is suitably manufactured from a material which is stiff enough to allow advancement of the device towards the site of treatment but which is also flexible enough to allow tracking of the device within the lumen of the endoscope, where endoscopic delivery is used.

In all embodiments of the invention, the penetrating member is suitably manufactured from stainless steel or Nitinol. Polyether ether ketone (PEEK), carbon fibre loaded liquid crystalline polymer, tungsten carbide or polyimide can also be used.

The electrodes of all embodiments of the invention are suitably constructed from a biocompatible metal such as stainless steel, platinum, silver, titanium, gold, a suitable alloy, and/or a shape memory alloy. The distance between the bipolar electrodes will, to an extent, define the shape of the thermal energy (in terms of embodiments relating to RF), ultrasound, microwave or IRE energy delivery patterns and the extent of the penetration of energy into the site of treatment. In the case of RF, greater separation between the electrodes tends to result in two distinct foci or regions of thermal energy, whereas closer spacing allows the areas of thermal energy to converge into a single elongated region. According to embodiments of the invention where the electrodes are connected to an RF generator, the distal and proximal bipolar electrodes are typically spaced no more than approximately 15 mm apart, and suitably between around 7 mm and about 10 mm or 12 mm apart.

In an embodiment of the invention, the device according to any previous embodiment can be configured so as to emit local radiotherapy, i.e. brachytherapy or internal radiotherapy, at the site of treatment. This is useful in instances where the site of therapy comprises or is close to a cancerous tumour, for example. By performing local radiotherapy, rather than external beam radiotherapy, the exposure of healthy tissue to radiation is significantly reduced. Local radiotherapy may be emitted from the device by providing a microwave or RF radiation source at or close to the distal tip of the device, or at any another suitable location on the device such as proximally to the most distal energy delivery element, e.g. electrode. Alternatively, an iridium-192 impregnated wire may be placed at or close to the tip of the device or may be located in a lumen of the device (if a lumen is present) or within a lumen of the penetrating member and exposed at or close to the tip of the device so as to emit local radiotherapy. Other suitable radio-isotopes may include caesium-137, cobalt-60, iodine-125, palladium-103 and ruthenium-106.

In any embodiment of the invention, the energy delivery elements, e.g. the electrodes 16 and 18, may be wider in diameter than the device to form a raised ring surface or they can be the same diameter as the device so that they are flush with the surface of the device.

In one embodiment of the invention an electrode is formed by simple exposure of a conducting element located within the core of the device. Hence, at specific regions of the device body a surface coating is removed (for example via laser etching) in order to expose the conducting element in the core of the device. In embodiments of the invention where the energy delivery element is located on an ultra-thin catheter (e.g. a catheter having a diameter of less than 3 French, <1 mm) or a guidewire, the electrode may also be formed at the distal tip by simple truncation of a covering insulating sleeve (such as a PTFE or PET coating) at a point proximally to the distal tip. Such an arrangement is demonstrated in FIGS. 22 (a) and (b).

The devices of the embodiments of the invention relating to FIGS. 1-21 and all alternative embodiments relating thereto, are suitably constructed as catheters in a variety of sizes typically ranging from about 0.15 mm up to about 3.3 mm in diameter (corresponds to French sizes 0.5 to 10). The lumen of the catheters should be large enough to accommodate a penetrating member of a size typically ranging from about 0.2 mm to about 2.0 mm or to accommodate an ablation device in the form of a narrow catheter or guidewire. The penetrating members may comprise a central lumen which is capable of accommodating a narrow catheter or guidewire of a diameter of up to about 0.6 mm (2 Fr). In specific embodiments of the invention the penetrating member may comprise a flexible hollow needle of gauges 19 (outer diameter (OD) 1.067 mm), 22 (OD 0.7176 mm) or 25 (OD 0.5144 mm).

Guidewires for use with devices of the invention are typically in the diameter size range of about 0.05 mm to about 1.2 mm, preferably about 0.20 mm to about 0.86 mm.

In an alternative embodiment of the invention, instead of using a twist-lock on the user control hub to advance and retract the penetrating member and to retain it in the desired position, it is possible to use other mechanisms such as a screw thread which extends from the external surface of the hub into the device (e.g. the lumen of the catheter) to retain the penetrating member in position. When the screw thread is loosened, the penetrating member can be advanced and retracted manually by the user. When the screw thread is tightened the penetrating member is retained in the desired position.

It should be understood that the different embodiments of the invention described herein can be combined where appropriate and that features of the embodiments of the invention can be used interchangeably with other embodiments where appropriate.

Although particular embodiments of the invention have been disclosed herein in detail, this has been done by way of example and for the purposes of illustration only. The aforementioned embodiments are not intended to be limiting with respect to the scope of the appended claims, which follow. It is contemplated by the inventors that various substitutions, alterations, and modifications may be made to the invention without departing from the spirit and scope of the invention as defined by the claims.

1-48. (canceled)

1. Apparatus suitable for causing tissue ablation at a specified therapeutic site in the body of a patient, comprising:
an endoscope having a distal end and a proximal end, wherein the endoscope comprises a first central lumen;
a catheter having a distal end and a proximal end and a second central lumen extending along its length;
an ablation device having a distal end and a proximal end, the distal end comprising at least one energy delivery element suitable for inducing tissue ablation; and,
wherein the catheter is capable of being coaxially positioned within the first central lumen of the endoscope and the ablation device is capable of being coaxially positioned within the second central lumen of the catheter, and wherein the catheter is capable of being advanced distally out of the first central lumen of the endoscope and retracted back to within the first central lumen of the endoscope, and the ablation device is capable of being advanced distally out of the second central lumen of the catheter and retracted back to within the second central lumen of the catheter.

2. The apparatus of claim 1, wherein the catheter comprises a penetrating member having a distal end comprising a sharp tip suitable for penetrating tissue.

3. The apparatus of claim 1, wherein the device is slidably positioned within the second central lumen of the catheter and the catheter is slidably positioned within the first central lumen of the endoscope.

4. The apparatus of claim 1, wherein the ablation device comprises a catheter, such as an ultra-thin catheter, or a guidewire.

5. The apparatus of claim 1, wherein the ablation device comprises an elongate body including a conductive core about which is located an insulating layer along at least a portion of the elongate body.

6. The apparatus of claim 1, wherein the at least one energy delivery element is located at the distal tip of the device.

7. The apparatus of claim 1, wherein the at least one energy delivery element is selected from the group consisting of: a monopolar radiofrequency electrode arrangement; a bipolar radiofrequency electrode arrangement; a plurality of radiofrequency electrodes; a microwave energy source; an ultrasound energy source; an irreversible electroporation energy source; and an electrical current energy source.

8. The apparatus of claim 1, wherein the at least one energy delivery element comprises a bipolar radiofrequency electrode arrangement, comprising a first electrode located at the distal end of the device and a second electrode located at a position proximally to the first electrode.

9. The apparatus of claim 1, wherein the catheter comprises at least one energy delivery element at its distal end.

10. The apparatus of claim 9, wherein the at least one energy delivery element is located at the distal tip of the catheter.

11. The apparatus of claim 10, wherein the at least one energy delivery element is selected from the group consisting of: a monopolar radiofrequency electrode arrangement; a bipolar radiofrequency electrode arrangement; a plurality of radiofrequency electrodes; a microwave energy source; an ultrasound energy source; an irreversible electroporation energy source; and an electrical current energy source.

12. The apparatus of claim 1, wherein the endoscope comprises an ultrasonic transducer at its distal end.

13. The apparatus of claim 1, wherein the apparatus comprises at least one enhanced ultrasound reflection surface.

14. The apparatus of claim 13, wherein the at least one enhanced ultrasound reflection surface is located on at least one of:
the surface of the device;
the surface of the at least one energy delivery element; and,
the surface of the catheter.

15. The apparatus of claim 1, wherein the device and/or catheter comprises means for emitting local radiotherapy at its distal end.

16. The apparatus of claim 15, wherein the means for emitting local radiotherapy comprises an iridium-192 impregnated member that is housed within the central lumen of the device or within a lumen or groove comprised within the catheter, and wherein the iridium-192 impregnated member can be advanced beyond the distal tip of the apparatus so as to expose the specified therapeutic site to local radiotherapy.

65-66. (canceled)

17. A method of ablating tissue at a specified therapeutic site in the body of a patient using the apparatus of claim 1, comprising:
advancing the endoscope along a hollow organ towards the site of treatment until the distal end of the endoscope is positioned close to the site of treatment;
advancing the distal end of the catheter out of the distal end of the endoscope so as to penetrate the tissue wall of the hollow organ;
advancing the distal end of the ablation device out of the distal end of the catheter and through the tissue wall of the hollow organ until the at least one heating element is located close to or at the site of treatment;
activating the at least one energy delivery element to cause tissue ablation at the site of treatment; and,
withdrawing the apparatus from the body after tissue ablation is complete.
